(19) United States
(12) Patent Application Publication
(10) Pub. No.: US 2010/0218278 A1
(43) Pub. Date: Aug. 26, 2010

(54) METHOD FOR IMPROVED STRESS TOLERANCE

(75) Inventors: Larry Von Kaster, JR., Ames, IA (US); Bruce A. Battles, Ames, IA (US); Wayne Alan Fithian, Elkhorn, NE (US); John Steffens, Chapel Hill, NC (US); Tracy L. Mader, Maple Grove, MN (US)

Correspondence Address: SYNGENTA BIOTECHNOLOGY, INC.;PATENT DEPARTMENT, 3054 CORNWALLIS ROAD, P.O. BOX 12257, RESEARCH TRIANGLE PARK, NC, 27709-2257, US

(73) Assignee: SYNGENTA PARTICIPATIONS AG, Research Triangle Park, NC (US)

(21) Appl. No.: 12/710,414

(22) Filed: Feb. 23, 2010

Related U.S. Application Data
(60) Provisional application No. 61/154,836, filed on Feb. 24, 2009.

(51) Int. Cl. A01H 1/00; A01H 5/00

(52) U.S. Cl. 800/279; 800/301

(57) ABSTRACT

A method of improving the vigor or yield of a crop plant is disclosed herein. The method involves introgressing a transgenic event comprising transgenes that encode an insecticidal protein and selectable marker protein into a crop plant, wherein the transgenic event and/or a protein thereof modulates in a transgenic crop plant tolerance to at least one abiotic stress condition when grown in a location essentially free of an insect pest population that is susceptible to the insecticidal protein.

### FIELD OF THE INVENTION

The present invention relates to compositions and methods for producing plants with improved biotic and abiotic stress tolerance.

### BACKGROUND

Both biotic and abiotic stresses are major factors limiting crop plant growth and productivity. Crop losses and reduction in yield of major crops including, among others, corn, wheat, soybean and rice caused by such stresses represent significant economic issues. Specifically, over 60% of the crop loss for the last 50 years has been due to abiotic stresses and such losses can total more than one billion dollars per year. Abiotic stresses include, among others, drought, heat, high plant population density, limited nitrogen, field flooding, cold temperatures, and field variability. Biotic stresses include, among others, insects, nematodes and pathogens such as fungi. All of these types of crop stress reduce growth and development and reduce yield.

The development of biotic and abiotic stress tolerant plants has the potential to mitigate or solve at least some of these problems. Creation of plants with tolerance to both biotic and abiotic stresses requires that multiple traits be stacked in the same crop plant. The use of traditional plant breeding strategies to produce such plants that exhibit tolerance to these types of stresses is slow and difficult. New molecular stacking technology improves the chances of success. However, even molecular stacking typically uses one transgene for one trait type. For example, a gene conferring drought tolerance will typically not confer insect resistance, and a gene conferring insect resistance will not typically confer drought tolerance in the absence of insect pressure. Having to stack many different transgenes in one plant limits the success of both traditional breeding and that of genetic engineering approaches to develop crop plants with both biotic and abiotic stress tolerance. It would be beneficial to identify genes and proteins involved in controlling the complex processes leading to both biotic and abiotic stress tolerance, for example without limitation, insect and drought stress tolerance.

Transgenic events comprising transgenes encoding insecticidal proteins which confer one type of trait, for example, insect resistance, can be grown economically only in production environments having yield-limiting infestations of pest insects susceptible to the insecticidal protein component of the transgenic event. For example, a grower would not typically plant an insect resistant transgenic crop in a production environment where there was little or no known yield limiting pest insect pressure. Therefore, such insect resistant crops have a limited number of production environments in which they can be used effectively.

There remains a continuing need to develop a better understanding of crop plant stress responses, so that corresponding methods can be developed to confer advantageous properties to crop plants. This need extends to the production of crops that exhibit resistance to damage by abiotic stress conditions such as drought, heat, high plant population density, limited nitrogen, field flooding, and cold temperatures. Even incremental gains in plant stress tolerance may have a significant economic impact in stabilizing the quality and supply of grain, oilseed and other harvested crop plant product. There remains also a need to develop stacked multi-trait transgenic crop plants wherein a transgene used in the stack confers more than one trait type on the transgenic crop plant.

### SUMMARY

In view of the above-identified needs and problems, the invention provides improved methods and compositions for developing and deploying insect resistant crop plants with improved tolerance to at least one abiotic stress condition.

The invention further relates to transgenic insect resistant crop plants that also provide tolerance to an abiotic stress even in the absence of insect pressure and to methods of improving vigor and yield of such transgenic crop plants. Such transgenic crop plants have introgressed into their genome at least one transgenic event that comprises a transgene encoding an insecticidal protein and another transgene encoding a selectable marker protein. Presence of the transgenic event or a protein thereof in a transgenic crop plant provides methods of improving the vigor or yield of the transgenic crop plant. By way of example, a method of the invention is useful to improve plant vigor or yield in geographic areas or with cultivation practices, where the particular transgenic crop plant is not normally used. Thus, the invention additionally provides a means to increase the number of production environments that the crop plant may be grown and improves the vigor or yield of the transgenic crop plant grown on such expanded production environments. The invention further provides events or proteins thereof that modulate in a transgenic crop plant tolerance to abiotic stresses, for example without limitation drought, cold, freezing, heat, salinity, high population density and soil nutrient deficiencies, in the absence of yield-limiting pest insect pressure. Such proteins are useful in providing multiple trait types and reducing the number of transgenes required for a stacked multi-trait transgenic product.

Field trials have confirmed the value of controlling insect pests for protecting yield of crop plants grown under abiotic stress conditions. For example without limitation, transgenic corn plants comprising a transgenic event that confers resistance to a root feeding insect pest have consistently higher yields than control corn plants when grown in drought stressed environments under moderate to severe insect pest feeding pressure.

According to the invention, it has unexpectedly been discovered that transgenic events comprising certain insecticidal proteins and selectable marker proteins can also confer improved vigor or yield on transgenic crop plants grown under abiotic stress conditions even in the absence of insect pests that are susceptible to the insecticidal protein. Thus, the events and/or proteins of the invention provide to a transgenic crop plant both increased resistance to insects and tolerance to abiotic stresses, for example without limitation, drought, cold stress, heat, salinity, high population density and soil nutrient deficiencies stress.

In one aspect, the invention provides a method of improving vigor or yield of a crop plant comprising introgressing a transgenic event into a crop plant resulting in a transgenic crop plant, wherein the transgenic event comprises a first transgene encoding an insecticidal protein and a second transgene encoding a selectable marker protein, and wherein the transgenic event and/or a protein thereof modulates in the transgenic crop plant tolerance to at least one abiotic stress condition compared to a control plant, and growing the transgenic crop plant at a location that is essentially free of an insect pest population which is susceptible to the insecticidal protein and in which the abiotic stress condition is yield limiting to the control plant, thereby improving vigor or yield of the transgenic crop plant compared to the control plant.

In another aspect, the invention provides a method of increasing the number of production environments in which a commercial insect resistant transgenic crop plant can be grown comprising providing transgenic seed for the transgenic crop plant comprising a transgenic event, wherein the transgenic event comprises a first transgene encoding an insecticidal protein and a second transgene encoding a selectable marker protein, and wherein the transgenic event and/or a protein thereof modulates in the transgenic crop plant tolerance to at least one abiotic stress condition compared to a control plant, and advertising that the transgenic seed be planted in a production environment that is essentially free of an insect pest population which is susceptible to the insecticidal protein and in which the abiotic stress condition is yield limiting to a control crop plant, thereby increasing the number of production environments in which the commercial insect resistant transgenic crop plant is grown.

In another aspect, the invention provides a method for improving vigor or yield in a transgenic crop plant exposed to an abiotic stress condition comprising providing transgenic seed for the crop plant comprising a transgenic event, wherein the transgenic event comprises a first transgene encoding an insecticidal protein and a second transgene encoding a selectable marker protein, and wherein the transgenic event and/or a protein thereof modulates in the transgenic crop plant tolerance to the abiotic stress condition compared to a control crop plant, and treating the seed or the transgenic crop plant with thiamethoxam which, in combination with the transgenic crop plant, is effective to improve vigor or yield in the crop plant to a degree greater than would be expected due to either the thiamethoxam or the transgenic event alone.

In another aspect, the invention provides a transgenic crop plant comprising a transgenic event, wherein the transgenic event comprises a first transgene encoding an insecticidal protein and a second transgene encoding a selectable marker protein, and wherein the transgenic event and/or a protein thereof modulates in the transgenic crop plant tolerance to at least one abiotic stress condition compared to a control plant.

Other aspects and advantages of the invention will become apparent to those skilled in the art from a study of the following description of the invention and non-limiting examples.

### DEFINITIONS

For clarity, certain terms used in the specification are defined and presented as follows:

“Abiotic stress condition” means a state unfavorable for a crop plant caused by environmental nonliving factors, which adversely affects plant metabolism, growth and/or development. A plant under the stress condition typically shows reduced germination rate, retarded growth and development, reduced photosynthesis rate, and eventually leading to reduction in yield. Specifically, “drought stress” means sub-optimal conditions for water and humidity needed for normal growth of natural plants. Relative water content (RWC) is one physiological measure of drought stress. RWC measures the effect of osmotic adjustment in plant water status, when a plant is under stressed conditions. RWC can result from drought stress as well as heat and induced osmotic stress.

As used herein, the term “advertising” means the personal or non-personal communication of information usually designed to influence purchasing behavior or thought patterns about products, services or ideas by identified sponsors through various media. Advertising is typically, but not exclusively done with signs, brochures, commercials, direct mailings, e-mail messages, or personal contact.

A “chimeric gene” is a recombinant nucleic acid sequence in which a promoter or regulatory nucleic acid sequence is operatively linked to a nucleic acid sequence that codes for an mRNA or which is expressed as a polypeptide, such that the regulatory nucleic acid sequence is able to regulate transcription or expression of the associated nucleic acid sequence. The regulatory nucleic acid sequence of the chimeric gene is not normally operatively linked to the associated nucleic acid sequence as found in nature.

A “coding sequence” is a nucleic acid sequence that is transcribed into RNA such as mRNA, rRNA, tRNA, snRNA, sense RNA or antisense RNA. Preferably the RNA is then translated in an organism to produce a protein.

To “control” insects means to inhibit, through a toxic effect, the ability of insect pests to survive, grow, feed, and/or reproduce, or to limit insect-related damage or loss in crop plants. To “control” insects may or may not mean killing the insects, although it preferably means killing the insects.

“Control plant” is a plant without a trait-improving transgenic event comprising a transgene of the invention. A control plant is used to measure and compare trait improvement in a transgenic plant with such trait-improving event. One suitable control plant is a non-transgenic plant of the parental line that was used to generate a transgenic plant. Another suitable control plant is a transgenic plant that comprises recombinant DNA without the specific trait producing transgene. Another suitable control plant is a negative segregant progeny of hemizygous transgenic plant. To make direct comparisons of a transgenic plant of the invention and control plants the transgenic and control plants are grown under the same conditions and at the same developmental stage.

As used herein, the term “corn” means Zea mays or maize and includes all plant varieties that can be bred with corn, including wild maize species.

The term “crop plant” means a plant of which a part or all is, or has been, harvested or cultivated on a commercial scale, or serves as an important source of food, feed, fiber or other chemical compounds. Without limitation, some examples of crop plants include, corn, wheat, barley, oats, rye, rice, vegetables, sugar cane, sugar beets, soybean, cotton and canola.

In the context of the invention the words “Cry protein” means a delta-endotoxin found naturally in Bacillus thuringiensis (Bt). Such Cry protein may be encoded by a synthetic DNA sequence comprised in a transgenic event.

The term “essentially free” of an insect pest population means an environment or location where the pest pressure from insects is significantly below a level which would cause any detriment to the plant's growth or yield due to the pest pressure; i.e. the pest population density is below the lowest pest population density that will cause economic damage to the crop. For example, without limitation, it is a common practice in the agricultural industry for crop fields such as corn to be scouted for pest populations present in the crop field (e.g. by taking a soil sample and counting the number of a specific pest insect). After such scouting is completed the skilled person can determine whether the pest population density is significant enough to warrant the application of a pesticide or the planting of seed of a transgenic insecticidal plant or whether the location in which the crop is or will be growing is essentially free of a particular pest and would not warrant the application of a pesticide or transgenic insecticidal plant for preventing or controlling injury to the crop by the particular insect pest i.e. the insect pest population is below the treatment threshold or economic injury level. Some insect pest populations may be scouted in the year previous to when control measures would be required by determining the presence of adult insects. For example, one economic threshold for corn rootworm is to recommend or apply corn rootworm control measure the year following scouting if an average of 0.75 beetles per plant are found during any of three field samplings done in August-mid September. The 0.75 beetles per plant threshold is based on 24,000 plants per acre (18,000 rootworm beetles per acre) and assumes a 50:50 ratio of males to females. Thus, a grower can determine if production environments or locations are essentially free of corn rootworm by taking soil samples and determining if larvae are present or by sampling adults in the previous year at that location. FIG. 1 shows the yield of control corn hybrids grown at locations having heavy or moderate corn rootworm pressure compared to locations that are essentially free of corn rootworm pressure. At locations having heavy corn rootworm pressure the yield is about 130 bu/A, whereas at locations that are essentially free of corn rootworm pressure, the yield is about 200 bu/A. Thus, yields may be about 54% higher in locations essentially free of corn rootworm pressure.

A transgenic “event” is produced by transformation of plant cells with heterologous DNA, i.e., a transgene comprising a coding sequence of interest, regeneration of a population of plants resulting from the insertion of the transgene into the genome of the plant, and selection of a particular plant characterized by insertion into a particular genome location. The term “event” refers to the original transformant and progeny of the transformant that include the heterologous DNA. The term “event” also refers to progeny produced by a sexual outcross between the transformant and another variety that include the heterologous DNA. Even after repeated back-crossing to a recurrent parent, the inserted DNA and flanking DNA from the transformed parent is present in the progeny of the cross at the same chromosomal location. The term “event” also refers to DNA from the original transformant comprising the inserted DNA and flanking genomic sequence immediately adjacent to the inserted DNA that would be expected to be transferred to a progeny that receives inserted DNA including the transgene of interest as the result of a sexual cross of one parental line that includes the inserted DNA (e.g., the original transformant and progeny resulting from selfing) and a parental line that does not contain the inserted DNA.

“Event MIR604” or “MIR604 event” or “MIR604” means a transgenic corn event, disclosed in U.S. Pat. No. 7,361,813 that has incorporated into its genome a cry3A055 transgene, disclosed in U.S. Pat. No. 7,230,167, and a pmi transgene, disclosed in U.S. Pat. No. 5,767,378. Therefore, MIR604 comprises a first transgene encoding a Cry3A055 insecticidal protein, useful in controlling corn rootworm (Diabrotica spp.) insect pests, and a second transgene encoding a phosphomannose isomerase enzyme (PMI), useful as a selectable marker, which allows a corn plant to utilize mannose as a carbon source.

A “gene” is a defined region that is located within a genome and that, besides the aforementioned coding nucleic acid sequence, comprises other, primarily regulatory, nucleic acid sequences responsible for the control of the expression, that is to say the transcription and translation, of the coding portion. A gene may also comprise other 5′ and 3′ untranslated sequences and termination sequences. Further elements that may be present are, for example, introns. A “transgene” is a gene heterologous to an organism into which it has been introduced by artificial techniques or natural techniques such as breeding.

“Genotype” as used herein is the genetic material inherited from parent plants not all of which is necessarily expressed in descendant plants. For example, without limitation, the MIR604 genotype refers to the heterologous genetic material transformed into the genome of a plant as well as the genetic material flanking the inserted sequence.

As used herein, the term “grower” means a person or entity that is engaged in agriculture, raising living organisms, such as crop plants, for food or raw materials.

A “heterologous” nucleic acid sequence is a nucleic acid sequence not naturally associated with a host cell into which it is introduced, including non-naturally occurring multiple copies of a naturally occurring nucleic acid sequence.

“Identity” or “percent identity” refers to the degree of similarity between two nucleic acid or protein sequences. For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2: 482 (1981), by the homology alignment algorithm of Needleman & Wunsch, J. Mol. Biol. 48: 443 (1970), by the search for similarity method of Pearson & Lipman, Proc. Nat'l. Acad. Sci. USA 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally, Ausubel et al., infra).

One example of an algorithm that is suitable for determining percent sequence identity and sequence similarity is the BLAST algorithm, which is described in Altschul et al., J. Mol. Biol. 215: 403-410 (1990). Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., 1990). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89: 10915 (1989)).

In addition to calculating percent sequence identity, the BLAST algorithm also performs a statistical analysis of the similarity between two sequences (see, e.g., Karlin & Altschul, Proc. Nat'l. Acad. Sci. USA 90: 5873-5787 (1993)). One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is less than about 0.1, more preferably less than about 0.01, and most preferably less than about 0.001.

Another widely used and accepted computer program for performing sequence alignments is CLUSTALW v1.6 (Thompson, et al. Nuc. Acids Res., 22: 4673-4680, 1994). The number of matching bases or amino acids is divided by the total number of bases or amino acids, and multiplied by 100 to obtain a percent identity. For example, if two 580 base pair sequences had 145 matched bases, they would be 25 percent identical. If the two compared sequences are of different lengths, the number of matches is divided by the shorter of the two lengths. For example, if there were 100 matched amino acids between a 200 and a 400 amino acid proteins, they are 50 percent identical with respect to the shorter sequence. If the shorter sequence is less than 150 bases or 50 amino acids in length, the number of matches are divided by 150 (for nucleic acid bases) or 50 (for amino acids), and multiplied by 100 to obtain a percent identity.

Another indication that two nucleic acids are substantially identical is that the two molecules hybridize to each other under stringent conditions. The phrase “hybridizing specifically to” refers to the binding, duplexing, or hybridizing of a molecule only to a particular nucleotide sequence under stringent conditions when that sequence is present in a complex mixture (e.g., total cellular) DNA or RNA. “Bind(s) substantially” refers to complementary hybridization between a probe nucleic acid and a target nucleic acid and embraces minor mismatches that can be accommodated by reducing the stringency of the hybridization media to achieve the desired detection of the target nucleic acid sequence.

When the subject method is described herein as “improving yield” of a crop plant, what is meant is that the yield of a product of the crop plant is increased by a measurable amount over the yield of the same product of a control plant produced under the same conditions. The yield may be increased by at least about 0.5%, or at least about 1%, or at least about 2%, or at least about 4%, or more. By way of example, if a transgenic corn hybrid of the invention yielded 162 bu/A and a control corn hybrid yielded 151 bu/A under the same growing conditions, then the yield of the transgenic corn hybrid would be said to have improved by ((162−151)/151)×100=5.9%.

As used herein, “insecticidal” means a toxic biological activity capable of controlling insects, preferably by killing them.

“Introgressing” a transgenic event means integration of the event into a non-transgenic crop plant or a transgenic crop plant not comprising an event or transgene of the invention by processes known in the art, for example without limitation, by backcross breeding methods or forward breeding methods. The result of introgressing a transgenic event into a crop plant is a transgenic crop plant.

An “isolated” nucleic acid molecule or protein is a nucleic acid molecule or protein that, by the hand of man, exists apart from its native environment and is therefore not a product of nature. An isolated nucleic acid molecule or protein may exist in a purified form or may exist in a non-native environment such as, for example without limitation, a recombinant microbial cell, plant cell, plant tissue, or plant. Thus, a nucleic acid found in nature that is removed from its native environment and transformed into a plant is still considered “isolated” even when incorporated into the genome of the resulting transgenic plant.

The term “modified Cry protein” means a Bt Cry protein-derived insecticidal protein having at least one additional protease recognition site that is recognized by a gut protease of a target insect, which does not naturally occur in the native Bt Cry protein. A modified Cry protein is not naturally occurring and, by the hand of man, comprises an amino acid sequence that is not identical to a naturally occurring Cry protein found in Bacillus thuringiensis.

As used herein, “modulates” refers to a change in activity (biological, chemical, or immunological) or lifespan resulting from specific binding between a molecule and either a nucleic acid molecule or a protein or resulting from a position effect of a transgenic event.

As used herein, the term “native gene” refers to a gene that is present in the genome of an untransformed cell or a gene that was present in the genome of a cell prior to transformation.

“Operatively linked” or “operably linked” refers to two nucleic acid sequences that are related physically or functionally. For example, a promoter or regulatory DNA sequence that is “operatively linked” or “operably linked” to a DNA sequence that codes for RNA or a polypeptide is intended to mean that a nucleic acid sequence encoding the polypeptide is linked to a regulatory sequence and termination region which allows expression in a plant cell. A typical construct consists, in the 5′ to 3′ direction of a regulatory region complete with a promoter capable of directing expression in a plant, RNA or polypeptide coding region, and a transcription termination region functional in plant cells. These constructs may be prepared in accordance with methodology well known to those of skill in the art of molecular biology (see for example: Sambrook et at. (1990), Molecular Cloning, 2nd ed. Cold Spring Harbor Press). The preparation of constructs may involve techniques such as restriction digestion, ligation, gel electrophoresis, DNA sequencing and PCR. A wide variety of cloning vectors is available to perform the necessary cloning steps.

“Plant material” refers to leaves, stems, roots, flowers or flower parts, fruits, pollen, egg cells, zygotes, seeds, cuttings, cell or tissue cultures, or any other part or product of a plant.

A “polypeptide” is an amino acid sequence comprising a plurality of consecutive polymerized amino acid residues e.g., at least about 15 consecutive polymerized amino acid residues, optionally at least about 30 consecutive polymerized amino acid residues, at least about 50 consecutive polymerized amino acid residues. In many instances, a polypeptide comprises a polymerized amino acid residue sequence that is a transcription factor or a domain or portion or fragment thereof. Additionally, the polypeptide may comprise 1) a localization domain, 2) an activation domain, 3) a repression domain, 4) an oligomerization domain, or 5) a DNA-binding domain, or the like. The polypeptide optionally comprises modified amino acid residues, naturally occurring amino acid residues not encoded by a codon, non-naturally occurring amino acid residues.

“Position effect” means a situation in which the phenotype expressed by a gene, either a native gene or a transgene, is altered by changes in the position of the gene within the plant genome. Expression of native genes may also be altered by position effects of an inserted transgene.

“Protein” refers to an amino acid sequence, oligopeptide, peptide, polypeptide or portions thereof whether naturally occurring or synthetic.

“Stringent hybridization conditions” and “stringent hybridization wash conditions” in the context of nucleic acid hybridization experiments such as Southern and Northern hybridizations are sequence dependent, and are different under different environmental parameters. Longer sequences hybridize specifically at higher temperatures. An extensive guide to the hybridization of nucleic acids is found in Tijssen (1993) Laboratory Techniques in Biochemistry and Molecular Biology-Hybridization with Nucleic Acid Probes part I chapter 2 “Overview of principles of hybridization and the strategy of nucleic acid probe assays” Elsevier, New York. Generally, highly stringent hybridization and wash conditions are selected to be about 5° C. lower than the thermal melting point (Tₘ) for the specific sequence at a defined ionic strength and pH. Typically, under “stringent conditions” a probe will hybridize to its target subsequence, but to no other sequences.

The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for hybridization of complementary nucleic acids which have more than 100 complementary residues on a filter in a Southern or northern blot is 50% formamide with 1 mg of heparin at 42° C., with the hybridization being carried out overnight. An example of highly stringent wash conditions is 0.1 5M NaCl at 72° C. for about 15 minutes. An example of stringent wash conditions is a 0.2×SSC wash at 65° C. for 15 minutes (see, Sambrook, infra, for a description of SSC buffer). Often, a high stringency wash is preceded by a low stringency wash to remove background probe signal. An example medium stringency wash for a duplex of, e.g., more than 100 nucleotides, is 1×SSC at 45° C. for 15 minutes. An example low stringency wash for a duplex of, e.g., more than 100 nucleotides, is 4-6×SSC at 40° C. for 15 minutes. For short probes (e.g., about 10 to 50 nucleotides), stringent conditions typically involve salt concentrations of less than about 1.0 M Na ion, typically about 0.01 to 1.0 M Na ion concentration (or other salts) at pH 7.0 to 8.3, and the temperature is typically at least about 30° C. Stringent conditions can also be achieved with the addition of destabilizing agents such as formamide. In general, a signal to noise ratio of 2× (or higher) than that observed for an unrelated probe in the particular hybridization assay indicates detection of a specific hybridization. Nucleic acids that do not hybridize to each other under stringent conditions are still substantially identical if the proteins that they encode are substantially identical. This occurs, e.g., when a copy of a nucleic acid is created using the maximum codon degeneracy permitted by the genetic code.

The following are examples of sets of hybridization/wash conditions that may be used to clone homologous nucleotide sequences that are substantially identical to reference nucleotide sequences of the present invention: a reference nucleotide sequence preferably hybridizes to the reference nucleotide sequence in 7% sodium dodecyl sulfate (SDS), 0.5 M NaPO₄, 1 mM EDTA at 50° C. with washing in 2×SSC, 0.1% SDS at 50° C., more desirably in 7% sodium dodecyl sulfate (SDS), 0.5 M NaPO₄, 1 mM EDTA at 50° C. with washing in 1×SSC, 0.1% SDS at 50° C., more desirably still in 7% sodium dodecyl sulfate (SDS), 0.5 M NaPO₄, 1 mM EDTA at 50° C. with washing in 0.5×SSC, 0.1% SDS at 50° C., preferably in 7% sodium dodecyl sulfate (SDS), 0.5 M NaPO₄, 1 mM EDTA at 50° C. with washing in 0.1×SSC, 0.1% SDS at 50° C., more preferably in 7% sodium dodecyl sulfate (SDS), 0.5 M NaPO₄, 1 mM EDTA at 50° C. with washing in 0.1×SSC, 0.1% SDS at 65° C.

A further indication that two nucleic acids or proteins are substantially identical is that the protein encoded by the first nucleic acid is immunologically cross reactive with, or specifically binds to, the protein encoded by the second nucleic acid. Thus, a protein is typically substantially identical to a second protein, for example, where the two proteins differ only by conservative substitutions.

The term “tolerance or tolerant to cold stress” is intended to describe a plant or plants which perform more favorably in any aspect of their growth and development under sub-optimal-reduced temperature conditions than do suitable control plants in the same conditions.

The term “tolerance or tolerant to freezing stress” is intended to describe a plant or plants which perform more favorably in any aspect of their growth and development under temperature conditions of less than or equal to 0 degrees C., than do suitable control panels in the same conditions.

The term “tolerance or tolerant to heat stress” is intended to describe a plant or plants which perform more favorably in any aspect of their growth and development under sub-optimal elevated temperature conditions than do suitable control plants in the same conditions.

The term “tolerance or tolerant to population density stress” is intended to describe a plant or plants which perform more favorably in any aspect of their growth and development or yield under a population planting density that would reduce yield in suitable control plants in the same conditions. For example, without limitation, typical plant population density for corn is about 29,000-34,000 plants per acre. At 38,000 plants per acre, when plants are under high population density stress, yield loss in control plants may be up to 35 bu/acre.

The term “tolerance or tolerant to salinity” is intended to describe a plant or plants which perform more favorably in any aspect of their growth and development under sub-optimal elevated salinity conditions than do suitable control plants in the same conditions.

With reference to the methods of the invention, a plant with increased tolerance to abiotic stress refers to a plant which performs more favorably in any aspect of growth and development under stress conditions than does a control plant of the same plant species grown under the same conditions.

“Transformation” is a process for introducing heterologous nucleic acid into a host cell or organism. In particular, “transformation” means the stable integration of a DNA molecule into the genome of an organism of interest.

“Transformed, transgenic or recombinant” refer to a host organism such as a plant into which a heterologous nucleic acid molecule has been introduced. The nucleic acid molecule is typically stably integrated into the genome of the host. Transformed cells, tissues, or plants are understood to encompass not only the end product of a transformation process, but also transgenic progeny thereof.

Thus, transgenic plants include progeny plants of an original plant derived from a transformation process including progeny of breeding transgenic plants with wild type plants or other transgenic plants. The enhancement of a desired trait can be measured by comparing the trait property in a transgenic plant which has recombinant DNA conferring the trait to the trait level in a progenitor plant. As used herein “progenitor plant” refers to a plant of essentially the same genotype as a transgenic plant but lacking the specific trait-conferring, transgene that characterizes the transgenic plant. Such a progenitor plant that lacks that transgene can be a natural, wild-type plant, an elite, non-transgenic plant, or a transgenic plant without the specific trait-conferring, transgene that characterizes the transgenic plant. The progenitor plant lacking the specific, trait-conferring transgene can be a sibling of a transgenic plant having the specific, trait-conferring transgene. Such a progenitor sibling plant may comprise other recombinant DNA.

“Vigor” means improved plant growth and development at the stages of seed imbibition through early vegetative phase, and improved plant growth and development at the stages of leaf development, flower production and seed maturity.

As used herein “water deficit” is a plant condition characterized by water potential in a plant tissue of less than −0.7 megapascals (MPa), e.g. −0.8 Mpa. Water potential in corn is conveniently measured by clamping a leaf segment in a pressurizable container so that a cut cross section of leaf is open to atmospheric pressure. Gauge pressure (above atmospheric pressure) on the contained leaf section is increased until water begins to exude from the atmospheric-pressure-exposed cross section; the gauge pressure at incipient water exudation is reported as negative water potential in the plant tissue, e.g. 7 bars of gauge pressure is reported as −0.7 MPa water potential. Water deficit can be induced by withholding water from plants for sufficient time that wild type plants are deleteriously affected, e.g. as manifested by reduced yield, stunted growth, retarded development, death or the like. The plants of this invention show a remarkable risibility after periods of water deficit that are adverse to control plants.

The term “water-use efficiency” refers to the ability of a plant to grow with substantially no yield penalty under extended periods with less than normal (typically about half) amounts of water.

As used herein “yield” of a transgenic crop plant of the invention can be measured in a number of ways, including test weight, seed number per plant, seed weight, seed number per unit area (i.e. seeds, or weight of seeds, per acre), bushels per acre, tones per acre, tons per acre, kilo per hectare. For example, corn yield may be measured as production of shelled corn kernels per unit of production area, e.g., in bushels per acre or metric tons per hectare, often reported on a moisture adjusted basis, e.g., at 15.5% moisture. Increased yield may result from enhanced utilization of key biochemical compounds, such as nitrogen, phosphorous and carbohydrate, or from improved responses to abiotic stresses, such as cold, heat, drought and salt. Recombinant DNA used in this invention can also be used to provide plants having enhanced growth and development, and ultimately increased yield, as the result of modified expression of plant growth regulators or modification of cell cycle or photosynthesis pathways.

As used herein, “yield potential” is the maximum production of a crop plant that can be achieved in a given production environment.

The nomenclature used herein for DNA bases and amino acids is as set forth in 37 C.F.R. §1.822.

### DETAILED DESCRIPTION

Field trials carried out in abiotic stressed production environments have confirmed the value of transgenic insect control, for protecting yield under such abiotic stress conditions. For example, transgenic corn rootworm-resistant corn plants comprising the corn event MIR604, expressing a modified Cry3A insecticidal protein, have consistently higher yields than control corn plants when grown in drought stressed environments under moderate to severe corn rootworm pressure.

In accordance with the invention, it has been unexpectedly discovered that transgenic events comprising insecticidal protein, such as mCry3A and a selectable marker protein, such as PMI, can also confer beneficial health effects on transgenic crop plants grown under abiotic stress conditions even in the absence of insect pests that are susceptible to the insecticidal protein. Thus, the events of the invention or the proteins thereof provide to a transgenic crop plant both increased resistance to insects and tolerance to abiotic stresses, for example without limitation, drought, cold stress, heat, salinity, high population density and soil nutrient deficiencies stress. Use of the transgenic events of the invention comprising an insecticidal protein and a selectable marker protein of the invention results in increased vigor or yield, reduces the number of transgenes required for stacking multiple traits in the same transgenic plant, and provides a means for expanding the geographic range in which the transgenic crop plants may be grown to improve vigor or yield compared to a control plants without the transgenic event.

In one embodiment, the invention encompasses a method of improving vigor or yield of a crop plant comprising introgressing a transgenic event into a crop plant resulting in a transgenic crop plant, wherein the transgenic event comprises a first transgene encoding an insecticidal protein and a second transgene encoding a selectable marker protein, and wherein the transgenic event and/or the insecticidal protein and/or the selectable marker protein modulates in the transgenic crop plant tolerance to at least one abiotic stress condition compared to a control plant, and growing the transgenic crop plant or progeny thereof at a location that is essentially free of an insect pest population which is susceptible to the insecticidal protein and in which the abiotic stress condition is yield limiting to the control plant, thereby improving vigor or yield of the transgenic crop plant compared to the control plant.

In one aspect of the invention, the crop plant is a monocot or a dicot. In one embodiment of this aspect, the monocot crop plant is selected from the group consisting of corn, wheat, rye, oat, triticale, rice, barley, sugarcane, and turf grass. In another embodiment of this aspect, the dicot crop plant is selected from the group consisting of soybean, cotton, rapeseed, canola, vegetables, sunflower, tobacco, tomato, and a forage crop.

In another aspect of the invention, the insect pest is a coleopteran insect. In one embodiment of this aspect, the coleopteran insect is corn rootworm.

In still another aspect of the invention, the insecticidal protein is selected from the group consisting of a Bacillus thuringiensis (Bt) Cry protein, a modified Cry protein, an engineered hybrid Cry protein and a vegetative insecticidal protein. In one embodiments of this aspect, the Cry protein is a Cry3 protein, particularly a Cry3A or Cry3B protein, more particularly a Cry3Aa or a Cry3Bb protein. In another embodiment of this aspect, the modified Cry protein is a mCry3A protein.

In another aspect of the invention, the selectable marker protein confers a growth advantage to transformed plant cells over non-transformed plant cells. In one embodiment of this aspect, the selectable marker protein confers upon a transformed plant cell the ability to utilize a sugar as a carbon source. In another embodiment of this aspect, the selectable marker protein is a phosphomannose isomerase (PMI).

In another aspect of the invention, the transgenic event is MIR604.

In another aspect of the invention, the abiotic stress is selected from the group consisting of drought stress, cold stress, freezing stress, heat stress, salinity stress, high population density stress or low nitrogen stress. In one embodiment of this aspect, the abiotic stress is drought stress.

In yet another aspect of the invention, the transgenic crop plant has improved yield compared to the control crop plant. In one embodiment of this aspect, the crop plant is corn and the event is MIR604. In one aspect of this embodiment, the yield of the MIR604 corn is at least 3 bushels per acre higher than the control corn or at least 10 bushels per acre higher than the control corn. In another embodiment, the insecticidal protein is mCry3A and the selectable marker protein is phosphomannose isomerase (PMI).

In another embodiment, the invention encompasses a method of improving vigor or yield of a corn plant comprising introgressing event MIR604 into a corn plant resulting in a transgenic MIR604 corn plant, wherein the MIR604 event comprises a first transgene encoding a mCry3A insecticidal protein and a second transgene encoding a PMI selectable marker protein, and wherein the MIR604 event and/or mCry3A and/or PMI modulates in the transgenic corn plant tolerance to at least a drought stress condition compared to a control corn plant; and growing the transgenic MIR604 corn plant or progeny thereof at a location that is essentially free of a corn rootworm pest population which is susceptible to the mCry3A insecticidal protein and in which the drought stress condition is yield limiting to the control corn plant, thereby improving vigor or yield of the transgenic corn plant compared to the control corn plant.

In still another embodiment, the invention encompasses a method of increasing the number of production environments in which a commercial insect resistant transgenic crop plant can be grown comprising providing transgenic seed for the crop plant comprising a transgenic event, wherein the transgenic event comprises a first transgene encoding an insecticidal protein and a second transgene encoding a selectable marker protein, and wherein the transgenic event and/or the insecticidal protein and/or the selectable marker protein modulates in the transgenic crop plant tolerance to at least one abiotic stress condition compared to a control plant; and advertising that the transgenic seed be planted in a production environment that is essentially free of an insect pest population which is susceptible to the insecticidal protein and in which the abiotic stress condition is yield limiting to a control crop plant, thereby increasing the number of production environments in which the commercial insect resistant transgenic crop plant is grown.

In another aspect this embodiment, the insect pest is a coleopteran insect. In a further aspect, the coleopteran insect is corn rootworm.

In still another aspect of this embodiment, the insecticidal protein is selected from the group consisting of a Bacillus thuringiensis (Bt) Cry protein, a modified Cry protein, an engineered hybrid Cry protein and a vegetative insecticidal protein. In a further aspect, the Cry protein is a Cry3 protein, particularly a Cry3A or Cry3B protein, more particularly a Cry3Aa or a Cry3Bb protein. In another aspect, the modified Cry protein is a mCry3A protein.

In another aspect of this embodiment, the selectable marker protein confers a growth advantage to transformed plant cells over non-transformed plant cells. In one embodiment of this aspect, the selectable marker protein confers upon a transformed plant cell the ability to utilize a sugar as a carbon source. In another embodiment of this aspect, the selectable marker protein is a phosphomannose isomerase (PMI).

In another aspect of this embodiment, the transgenic event is MIR604 and the crop plant is corn.

In another aspect of this embodiment, the abiotic stress is selected from the group consisting of drought stress, cold stress, freezing stress, heat stress, salinity stress, high population density stress or low nitrogen stress. In one embodiment of this aspect, the abiotic stress is drought stress.

In another embodiment, the invention encompasses a method of increasing the number of production environments in which a commercial insect resistant transgenic corn plant can be grown comprising providing transgenic seed for the corn plant comprising event MIR604, wherein event MIR604 comprises a first transgene encoding a mCry3A insecticidal protein and a second transgene encoding a PMI selectable marker protein, and wherein event MIR604 and/or mCry3A and/or PMI modulates in the transgenic corn plant tolerance to at a least drought stress condition compared to a control plant; and advertising that the transgenic seed be planted in a production environment that is essentially free of a corn rootworm pest population which is susceptible to the mCry3A insecticidal protein and in which the drought stress condition is yield limiting to a control corn plant.

In one aspect of this embodiment the MIR604 corn plants allow farmers to produce good corn yields in production environments that tend to be dry and particularly in those dry areas essentially free of corn rootworm. The MIR604 corn plants also provide framers with the means to reduce the need for irrigation in a normal-rain growing season and in dry years.

Crop plants respond to abiotic stress by producing ethylene, a natural plant hormone. Ethylene produced under stress conditions triggers various negative responses including wilt or rolled leaves, firing of lower leaves, premature leaf death, reduced photosynthetic efficiency, chlorophyll loss, poor pollination and seed aboration, among others.

Research has shown that the chemical 1-Methylcycloprene (1-MCP) applied to a crop plant, prevents the ethylene signal from triggering stress responses. Such application of 1-MCP thus maintains active crop growth and development, resulting in a more robust crop, leading to increased yield. The chemical thiamethoxam has been shown to also improve the health characteristics of plants upon application to seeds or the crop plants themselves. Such beneficial health effects include faster emergence, improved plant stands, increased root mass, thicker stems, earlier canopies, taller greener plants, improved quality and higher yields.

A transgenic crop plant of the invention can also be treated with chemicals such as 1-MCP or thiamethoxam that in combination with the properties of the transgenic event comprised in the transgenic crop plant provide a method of further improving the yield potential of the transgenic crop plant grown in production environments under abiotic stress beyond that which would be expected from the application of 1-MCP alone or thiamethoxam alone or the transgenic event alone.

Thus, in one embodiment, the invention encompasses a method for improving vigor or yield in a transgenic crop plant exposed to an abiotic stress condition comprising providing transgenic seed for the crop plant comprising a transgenic event, wherein the transgenic event comprises a first transgene encoding an insecticidal protein and a second transgene encoding a selectable marker protein, and wherein the transgenic event and/or the insecticidal protein and/or the selectable marker protein modulates in the transgenic crop plant tolerance to the abiotic stress condition compared to a control crop plant, and treating a transgenic crop plant grown from the seed with 1-Methylcyclopropene (1-MCP) which, in combination with the transgenic crop plant, is effective to improve vigor or yield in the transgenic crop plant to a degree greater than would be expected due to either the 1-MCP or the transgenic event and/or the insecticidal protein and/or the selectable marker protein alone.

In another embodiment, the invention encompasses a method for improving vigor or yield in a transgenic crop plant exposed to an abiotic stress condition comprising providing transgenic seed for the crop plant comprising a transgenic event, wherein the transgenic event comprises a first transgene encoding an insecticidal protein and a second transgene encoding a selectable marker protein, and wherein the transgenic event and/or the insecticidal protein and/or the selectable marker protein modulates in the transgenic crop plant tolerance to the abiotic stress condition compared to a control crop plant, and treating a transgenic crop plant grown from the seed with thiamethoxam which, in combination with the transgenic crop plant, is effective to improve vigor or yield in the transgenic crop plant to a degree greater than would be expected due to either the thiamethoxam or the transgenic event and/or the insecticidal protein and/or the selectable marker protein alone.

In another embodiment, the invention encompasses a method for improving vigor or yield in a transgenic corn plant exposed to at least a drought stress condition comprising providing transgenic seed for the corn plant comprising event MIR604, wherein event MIR604 comprises a first transgene encoding a mCry3A insecticidal protein and a second transgene encoding a PMI selectable marker protein, and wherein the MIR604 event and/or mCry3A and/or PMI modulates in the transgenic corn plant tolerance to the drought stress condition compared to a control corn plant, and treating a transgenic corn plant grown from the seed with 1-Methylcyclopropene (1-MCP) which, in combination with the transgenic corn plant, is effective to improve vigor or yield in the corn plant exposed to the drought stress condition to a degree greater than would be expected due to either the 1-MCP alone or event MIR604 and/or mCry3A and/or PMI alone.

In still another embodiment, the invention encompasses a method for improving vigor or yield in a transgenic corn plant exposed to at least a drought stress condition comprising providing transgenic seed for the corn plant comprising a MIR604 event, wherein the MIR604 event comprises a first transgene encoding a mCry3A insecticidal protein and a second transgene encoding a PMI selectable marker protein, and wherein event MIR604 and/or mCry3A and/or PMI modulates in the transgenic corn plant tolerance to the drought stress condition compared to a control corn plant; and treating a transgenic corn plant grown from the seed with thiamethoxam which, in combination with the transgenic corn plant, is effective to improve vigor or yield in the transgenic corn plant exposed to the drought stress condition to a degree greater than would be expected due to either the thiamethoxam alone or event MIR604 and/or mCry3A and/or PMI alone. During the development of transgenic crop plants much effort is concentrated on optimization of the insertion and expression of the transgene in particular transgenic events, and then introgressing the transgene throughout the breeding population by classical breeding methods. The site of insertion of a transgene into the host genome has been a concern for at least two reasons; (i) the region where it inserted may modulate the level of expression of the transgene, and (ii) the insertion of the transgene may disrupt the normal function or expression of a gene near or where it has been inserted. The selection of genomic locations that are beneficial for gene integration provides for suitable levels of stable expression of an introduced gene, or genes, and generally does not negatively affect other agronomic characteristics of the crop plant.

The genomic region in which the transgene has been inserted also provides agronomic phenotypes to the crop plant. These phenotypes have their own value in a breeding program and these regions should be considered when selecting among multiple transgene insertion events. Transgene insertion events into genomic regions that are associated with improved performance with respect to an agronomic trait or multiple trait index result in an improved phenotype in the crop plant and progeny derived from the crop plant that contain the transgene and the associated improved phenotype. Selecting for the transgenic event necessarily results in selecting a segment of the host genome that surrounds it, and the improved phenotypic effect. Further improvements involve the identification of molecular markers for the tracking and maintenance of the genomic segment with the associated transgene. This is an area that has not been adequately addressed in current plant breeding with transgene insertion events.

### Plant Transformation

Events of the invention are created by transforming a plant with expression cassettes comprising genes that encode an insecticidal protein and a selectable marker. The coding portion of such genes can be optimized for expression in any plant. It is recognized that all or any part of the gene sequence may be optimized or synthetic. That is, synthetic or partially optimized sequences may also be used. Plants transformed in accordance with the invention may be monocots or dicots and include, but are not limited to, maize (corn), wheat, barley, rye, vegetables, soybean, sorghum, sugarcane, sugar beet, sunflower, rapeseed, cotton, alfalfa and rice.

Once a desired event has been identified, it may be introgressed into other varieties of the same plant species, particularly including commercial varieties, using traditional breeding techniques.

Gene expression in transgenic plants is driven by promoters that function in plants. The choice of promoter will vary depending on the temporal and spatial requirements for expression, and also depending on the target species. Thus, according to the invention, the nucleotide sequences of the invention are expressed in leaves, in stalks or stems, in ears, in inflorescences (e.g. spikes, panicles, cobs, etc.), in roots, and/or seedlings among others. In many cases, however, protection against more than one type of insect pest is required or multiple transgenes for multiple traits are required, and thus expression in multiple tissues is desirable. Although many promoters from dicotyledons have been shown to be operational in monocotyledons and vice versa, ideally dicotyledonous promoters are selected for expression in dicotyledons, and monocotyledonous promoters for expression in monocotyledons. However, there is no restriction to the provenance of selected promoters; it is sufficient that they are operational in driving the expression of the nucleotide sequences in the desired cell.

In accordance with the invention, constitutive promoters are active under most environmental conditions and states of development or cell differentiation. These promoters are likely to provide expression of the genes at many stages of plant development and in a majority of tissues. A variety of constitutive promoters are known in the art. Examples of constitutive promoters that are active in plant cells include but are not limited to the actin, ubiquitin, CaMV 35S and 19S promoters.

Tissue-specific promoters cause transcription or enhanced transcription of a polynucleotide sequence in specific cells or tissues at specific times during plant development, such as in vegetative or reproductive tissues. Examples of tissue-specific promoters under developmental control include promoters that initiate transcription primarily in certain tissues, such as vegetative tissues, e.g., roots, leaves or stems, or reproductive tissues, such as fruit, ovules, seeds, pollen, pistils, flowers, or any embryonic tissue, or any combination thereof. Reproductive tissue specific promoters may be, e.g., ovule-specific, embryo-specific, endosperm-specific, integument-specific, pollen-specific, petal-specific, sepal-specific, or some combination thereof. Tissue specific promoter(s) will also include promoters that can cause transcription, or enhanced transcription in a desired plant tissue at a desired plant developmental stage. An example of such a promoter includes, but is not limited to the metallothionein promoter (MTL), disclosed in U.S. Pat. No. 5,466,785, and PEPC or trpA promoters disclosed in U.S. Pat. No. 5,625,136. One skilled in the art will recognize that a tissue-specific promoter may drive expression of operably linked polynucleotide molecules in tissues other than the target tissue. Thus, as used herein, a tissue-specific promoter is one that drives expression preferentially not only in the target tissue, but may also lead to some expression in other tissues as well.

In practicing this invention, an inducible promoter may also be used to ectopically express the structural gene in the recombinant DNA construct. A inducible promoter is a promoter induced by a specific stimuli such as abiotic stress conditions comprising, for example, light, temperature, chemicals, drought, high salinity, osmotic shock or oxidant conditions and include, but is not limited to, the light-inducible promoter derived from the pea rbcS gene, the promoter from the alfalfa rbcS gene, the promoters DRE, MYC and MYB active in drought; the promoters INT, INPS, prxEa, Ha hsp17.7G4 and RD21 active in high salinity and osmotic stress.

Trangenes driven by the metallothionein (MTL) promoter are inducible by metal ions such as copper, zinc, by other chemical inducers and by abiotic stress. Seed treatments incorporating these inducers in various forms may be used to enhance expression and activity of a transgene encoding an insecticidal protein of the invention. Thus, an MTL promoter may function as both a tissue-preferred and inducible promoter.

In one embodiment, the invention encompasses a transgenic event wherein expression of one or more proteins of the invention is driven by an inducible metallothionein (MTL) promoter, including but not limited to the MTL promoter disclosed in U.S. Pat. No. 5,466,785. Such abiotic stress induced protein expression provides enhanced or improved modulation of abiotic stress tolerance in a transgenic crop plant. In one aspect of this embodiment, a transgenic event of the invention comprises a transgene wherein an MTL promoter is operably linked to a polynucleotide coding for a mCry3A insecticidal protein, wherein when the transgenic event is introgressed into a crop plant, the mCry3A modulates tolerance to an abiotic stress.

It may be preferable to target expression of the nucleotide sequences of the present invention to different cellular localizations in the plant. In some cases, localization in the cytosol may be desirable, whereas in other cases, localization in some subcellular organelle may be preferred. Subcellular localization of transgene-encoded enzymes is undertaken using techniques well known in the art. Typically, the DNA encoding the target peptide from a known organelle-targeted gene product is manipulated and fused upstream of the nucleotide sequence. Many such target sequences are known for the chloroplast and their functioning in heterologous constructions has been shown. The expression of the nucleotide sequences of the present invention is also targeted to the endoplasmic reticulum or to the vacuoles of the host cells. Techniques to achieve this are well known in the art.

Vectors suitable for plant transformation are described elsewhere in this specification. For Agrobacterium-mediated transformation, binary vectors or vectors carrying at least one T-DNA border sequence are suitable, whereas for direct gene transfer any vector is suitable and linear DNA containing only the construction of interest may be preferred. In the case of direct gene transfer, transformation with a single DNA species or co-transformation can be used (Schocher et al. Biotechnology 4:1093-1096 (1986)). For both direct gene transfer and Agrobacterium-mediated transfer, transformation is usually (but not necessarily) undertaken with a selectable marker that may provide resistance to an antibiotic (kanamycin, hygromycin or methotrexate) or a herbicide (basta). Plant transformation vectors comprising the modified Cry3A toxin genes of the present invention may also comprise genes (e.g. phosphomannose isomerase; PMI) which provide for positive selection of the transgenic plants as disclosed in U.S. Pat. Nos. 5,767,378 and 5,994,629, herein incorporated by reference. The choice of selectable marker is not, however, critical to the invention.

Generally, successful creation of transgenic events requires an efficient selection system. Genes that are frequently used to select transformed tissue include but are not limited to nptII, hph and bar or pat, coding for neomycin phosphotransferase, hygromycin phosphotransferase and phosphinothricin acetyl transferase, respectively. These so called negative selection genes confer resistance to kanamycin, to Hygromycin and to phosphinothricin. Transformed cells in these negative selection systems are able to to survive, while non-transformed cells are killed by the selective agent.

Other selection systems rely on gene products that provide a growth advantage to transformed plant cells over non-transformed plant cells. Such positive selection involves conferring onto the transformed cell a metabolic advantage such as the capability of utilizing a sugar as a carbon source, or other competitive advantages for stimulating cell growth over non-transformed cells such as response to hormone and adaptation to extreme temperature. One example of a positive selectable marker is phosphomannose isomerase (PMI), which allows transformed plant cells to utilize mannose as a carbon source. Mannose, a hexose sugar, strongly inhibits root growth and respiration in non-transformed plants and may inhibit seed germination. Therefore, plant cells transformed to express PMI acquire a growth advantage on mannose-containing media over non-transformed cells.

In one embodiment, the invention encompasses a transgene encoding a positive selectable marker protein, wherein the selectable marker protein modulates in a transgenic plant tolerance to at least one abiotic stress condition. In one aspect of this embodiment, the selectable marker is PMI.

In another embodiment, the invention encompasses a transgenic crop plant comprising a transgenic event, wherein the transgenic event comprises a first transgene encoding an insecticidal protein and a second transgene encoding a selectable marker protein, and wherein the transgenic event and/or the insecticidal protein and/or the selectable marker protein modulates in the transgenic crop plant tolerance to at least one abiotic stress condition compared to a control plant.

In one aspect of this embodiment, the crop plant is a monocot or a dicot. In a further aspect, the monocot crop plant is selected from the group consisting of corn, wheat, rye, oat, triticale, rice, barley, sugarcane, and turf grass. In another aspect of this embodiment, the dicot crop plant is selected from the group consisting of soybean, cotton, rapeseed, canola, vegetables, sunflower, tobacco, tomato, and a forage crop.

In still another aspect of this embodiment, the insecticidal protein is selected from the group consisting of a Bacillus thuringiensis (Bt) Cry protein, a modified Cry protein, an engineered hybrid Cry protein and a vegetative insecticidal protein. In a further aspect, the Cry protein is a Cry3 protein, particularly a Cry3A or Cry3B protein, more particularly a Cry3Aa or a Cry3Bb protein. In another aspect, the modified Cry protein is a mCry3A protein.

In another aspect of this embodiment, the selectable marker protein is a phosphomannose isomerase (PMI).

In another aspect of this embodiment, the transgenic event is MIR604 and the crop plant is corn.

In another aspect of this embodiment, the abiotic stress is selected from the group consisting of drought stress, cold stress, freezing stress, heat stress, salinity stress, high population density stress or low nitrogen stress. In a further aspect, the abiotic stress is drought stress.

In still another embodiment, the invention encompasses a transgenic crop plant comprising a recombinant polynucleotide encoding an insecticidal protein which has no significant activity against any insect pest for such crop plant, wherein the insecticidal protein modulates in the transgenic crop plant improved tolerance to at least one abiotic stress condition compared to a control plant.

### EXAMPLES

The invention, now being generally described, will be more readily understood by reference to the following examples, which are included merely for purposes of illustration of certain aspects and embodiments of the invention and are not intended to limit the invention.

### Example 1

### MIR604 Yield Compared to Control Corn Yield

This example illustrates the aspect of the invention relating to yield potential of transgenic corn. Hybrid seed of MIR604 transgenic corn is planted in multiple field trials to evaluate the yield potential of MIR604 corn compared to control corn plants at locations essentially free of corn rootworm pressure. All plants are maintained using standard agronomic practices from the time the seed is planted through harvest at which time yield is measured using standard industry practices.

The MIR604 hybrids had a yield of 189.8 bushels/A compared to the control hybrids that had 186.0 bushels/A. These results show that the MIR604 hybrid corn plants provide an overall increase in yield compared to control plants (non-MIR604 hybrid corn) even in locations essentially free of corn rootworm, which is a pest susceptible to the mCry3A protein expressed in the MIR604 hybrid corn.

### Example 2

### MIR604 Response to Drought Stress

This example illustrates another aspect of the invention relating to yield potential of transgenic corn under abiotic stress conditions. Hybrid seed of MIR604 transgenic corn is planted in field trials to evaluate its drought tolerance as compared to control corn hybrids at locations essentially free of corn rootworm pressure. The plants are grown in multiple locations including managed stress environments (MSE) in the US. Typically each MSE has little or no natural rainfall during the growing season, has precision irrigation capability, uniform temperature and soil types, essentially no insect and disease pressure, and high yield potential when irrigated. While non-MSE locations do not afford complete control of moisture to the corn plant, they provide yearly drought stress conditions representative of dryland corn production on millions of acres.

Ideally, irrigation should occur every 3-4 days with 10-20 cm of water at a time. The amount of water applied should depend upon the stage of the plant and weather conditions. Water is withheld from half of the planting at each location during the late vegetative stage approximately 30 days before flowering. The severity of drought stress should be appropriate to achieve about 40% yield reduction in stressed plants relative to well-watered plants.

The experimental evidence shows that under drought conditions transgenic MIR604 corn plants expressing mCry3A, under control of a stress inducible MTL promoter and the PMI protein under control of a ubiquitin promoter are healthier (i.e. have improved vigor) than the control plants and exhibit some or all of the following phenotypes: (a) likely to have a higher chlorophyll index, e.g. >42 in MIR604 plants as compared to <40 in control plants, (b) likely to produce more photosynthate, (c) likely to have cooler leaf temperature, and (d) likely to maintain higher stomatal conductance. The MIR604 hybrid corn plants also have increased yield compared to control hybrids.

A summary of yield results is shown in Table 1.

TABLE 1
Effects of drought stress on yield of MIR604 and control hybrids.

Yield (Bu/A)

| Hybrid/Location | MIR604 | Control |
|---|---|---|
| Hybrid 1/Gilroy, CA | 210.6 | 199.6 |
| Hybrid 2/Gilroy, CA | 201.6 | 188.1 |
| Hybrid 3/LaSalle, CO | 101.2 | 91.4 |
| Hybrid 4/LaSalle, CO | 136.3 | 128.2 |
| All Combined | 162.4 | 151.8 |

Under drought conditions MIR604 hybrid corn has an overall yield advantage over non-MIR604 hybrid corn of about 11 Bu/A at locations essentially free of corn rootworm pest population pressure. Therefore, the improved vigor and yield advantage under drought stress between MIR604 and non-MIR604 hybrids is attributed to the MIR604 event and/or the insecticidal protein and/or the selectable marker protein.

### Example 3

### MIR604 Response to High Population Density Stress

This example illustrates another aspect of the invention relating to yield potential of transgenic corn under abiotic stress conditions. Hybrid seed of MIR604 transgenic corn is planted in field trials to evaluate its tolerance to population density stress as compared to control corn hybrids at locations essentially free of corn rootworm pressure. The plants are grown in multiple locations including managed stress environments (MSE) in the US. Typically each MSE has little or no natural rainfall during the growing season, has precision irrigation capability, uniform temperature and soil types, essentially no insect and disease pressure, and high yield potential when irrigated. Hybrids are tested at the following densities: Low population density (26,000 plants/A); Normal density (32,000 plants/A); High density (38,000 plants/A) and Very High density (44,000 plants/A).

Yield results are shown in Table 2. Under High and Very High population density stress, MIR604 hybrid corn has an overall yield advantage over non-MIR604 hybrid corn. Therefore, the improved yield advantage under population density stress between MIR604 and non-MIR604 hybrids is attributed to the MIR604 event and/or the insecticidal protein and/or the selectable marker protein.

TABLE 2
Effects of population density on yield.

Yield (Bu/A)

| Population Density | MIR604 | Control |
|---|---|---|
| Low (26K plants/A) | 254.6 | 254.9 |
| Normal (32K plants/A) | 267.5 | 270.4 |
| High (38K plants/A) | 267.5 | 264.8 |
| Very High (44K plants/A) | 272.5 | 267.9 |

### Example 4

### MIR604 Response to Low Nitrogen Stress

This example illustrates another aspect of the invention relating to yield potential of transgenic corn under abiotic stress conditions. Hybrid seed of MIR604 transgenic corn is planted in field trials to evaluate its tolerance to nitrogen stress as compared to control corn hybrids at locations essentially free of corn rootworm pressure. The plants are grown in multiple locations including managed stress environments (MSE) in the US. Typically each MSE has little or no natural rainfall during the growing season, has precision irrigation capability, uniform temperature and soil types, essentially no insect and disease pressure, and high yield potential when irrigated. Hybrids are tested at the following nitrogen levels: Normal nitrogen input (Approx. 150 lb/A); Zero nitrogen input (0 lb/A) and Depleted nitrogen (Zero nitrogen input, depleted nitrogen in past 18 months, after planting 3 cover crops).

Yield results are shown in Table 3. Under High and Very High population density stress, MIR604 hybrid corn has an overall yield advantage over non-MIR604 hybrid corn. Therefore, the improved yield advantage under population density stress between MIR604 and non-MIR604 hybrids is attributed to the MIR604 event and/or the insecticidal protein and/or the selectable marker protein.

TABLE 3
Effects of nitrogen stress on yield.

Yield (Bu/A)

| Nitrogen Level | MIR604 | Control |
|---|---|---|
| Normal | 270.1 | 268.8 |
| No Nitrogen | 256.9 | 254.8 |
| Depleted Nitrogen | 209 | 202.8 |

The above examples illustrate practice of the invention. It will be appreciated by those skilled in the art that numerous variations and modifications may be made without departing from the spirit and scope of the invention.

1. A method of improving yield of a crop plant comprising:
a. introgressing a transgenic event into a crop plant resulting in a transgenic crop plant, wherein the transgenic event comprises a first transgene encoding an insecticidal protein and a second transgene encoding a selectable marker protein, and wherein the transgenic event and/or the insecticidal protein and/or the selectable marker protein modulates in the transgenic crop plant tolerance to at least one abiotic stress condition compared to a control plant; and
b. growing the transgenic crop plant or progeny thereof at a location that is essentially free of an insect pest population which is susceptible to the insecticidal protein and in which the abiotic stress condition is yield limiting to the control plant, thereby improving yield of the transgenic crop plant compared to the control plant.

2. The method of claim 1, wherein the crop plant is corn.

3. The method of claim 1, wherein the insecticidal protein is a Bacillus thuringiensis (Bt) Cry protein or a modified Cry protein.

4. The method of claim 3, wherein the Bacillus thuringiensis (Bt) Cry protein or modified Cry protein is active against a coleopteran insect.

5. The method of claim 4, wherein the coleopteran insect is a corn rootworm.

6. The method of claim 5, wherein the Cry protein is a Cry3 protein.

7. The method of claim 6, wherein the Cry3 protein is a Cry3A protein or a Cry3B protein.

8. The method of claim 7, wherein the Cry3A is a Cry3Aa or the Cry3B is a Cry3Bb.

9. The method of claim 5, wherein the modified Cry protein is a mCry3A protein.

10. The method of claim 1, wherein the selectable marker protein is a phosphomannose isomerase (PMI).

11. The method of claim 2, wherein the transgenic event is MIR604.

12. The method of claim 11, wherein the abiotic stress is selected from the group consisting of drought stress, cold stress, freezing stress, heat stress, salinity stress, high population density stress or low nitrogen stress.

13. The method of claim 12, wherein the yield of the MIR604 corn is at least 3 bushels per acre higher than the control corn.

14. The method of claim 13, wherein the yield of the MIR604 corn is at least 10 bushels per acre higher than the control corn.

15. A method of improving vigor or yield of a corn plant comprising:
a. introgressing event MIR604 into a corn plant resulting in a transgenic MIR604 corn plant, wherein the MIR604 event comprises a first transgene encoding a mCry3A insecticidal protein and a second transgene encoding a PMI selectable marker protein, and wherein the MIR604 event and/or mCry3A and/or PMI modulates in the transgenic corn plant tolerance to at least a drought stress condition, population density stress or nitrogen stress compared to a control corn plant; and
b. growing the transgenic MIR604 corn plant or progeny thereof at a location that is essentially free of a corn rootworm pest population which is susceptible to the mCry3A insecticidal protein and in which the drought stress condition, population density stress or nitrogen stress is yield limiting to the control corn plant, thereby improving vigor or yield of the transgenic corn plant compared to the control corn plant.

16. A transgenic corn plant comprising a transgenic event, wherein the transgenic event comprises a first transgene encoding an insecticidal protein and a second transgene encoding a selectable marker protein, and wherein the transgenic event and/or the insecticidal protein and/or the selectable marker protein modulates in the transgenic corn plant tolerance to at least one abiotic stress condition compared to a control plant.

17. The plant of claim 16, wherein the abiotic stress is selected from the group consisting of drought stress, cold stress, freezing stress, heat stress, salinity stress, high population density stress or low nitrogen stress.

18. The plant of claim 19, wherein the modified Cry protein is a mCry3A protein.

19. The plant of claim 18, wherein the transgenic corn plant has improved yield compared to the control crop plant grown under an abiotic stress condition.

20. The plant of claim 19, wherein the transgenic event is MIR604.
